(19) United States
(12) Patent Application Publication
(10) Pub. No.: US 2007/0232873 A1
(43) Pub. Date: Oct. 4, 2007

(54) NONINVASIVE, ACCURATE GLUCOSE MONITORING WITH OCT BY USING TISSUE WARMING AND TEMPERATURE CONTROL

(75) Inventors: Rinat O. Esenaliev, League City, TX (US); Donald S. Prough, Galveston, TX (US)

Correspondence Address: ROBERT W STROZIER, P.L.L.C, PO BOX 429, BELLAIRE, TX, 77402-0429, US

(73) Assignee: The Board of Regents of The University of Texas Syatem, Austin, TX (US)

(21) Appl. No.: 11/685,677

(22) Filed: Mar. 13, 2007

Related U.S. Application Data
(60) Provisional application No. 60/783,173, filed on Mar. 16, 2006.

(51) Int. Cl. A61B 5/00

(52) U.S. Cl. 600/316

(57) ABSTRACT

A new OCT system and method are disclosed, where the system includes a probe equipped with a heating element and a high heat conductive member to warm a tissue site to be scanned to an elevated and/or to maintain the elevated tissue temperature with a temperature variation of less than or equal to 1° C. to improve an accuracy and reliability of an OCT glucose concentration value other long measurement durations. The new OCT system and method can also be equipped with pressure components to reduce a pressure exerted on the tissue site to a minimal constant pressure.

### RELATED APPLICATIONS

This application claims priority to U.S. Provisional Application Ser. No. 60/783,173 filed 16 Mar. 2006.

### BACKGROUND OF THE INVENTION

1. Field of the Invention

The present invention relates to a method and system for continuous, noninvasive glucose monitoring in an animal including an human using an optical coherence tomography (OCT) based glucose monitoring system under conditions of a temperature of a tissue site and/or a pressure exerted on the tissue site sufficient to increase an accuracy of a calculated OCT glucose concentration.

More particularly, the present invention relates to a method for continuous noninvasive glucose monitoring in an animal including an human using a temperature and/or pressure controlled OCT based glucose monitoring system. The method includes the step of generating radiation. A first portion of radiation is directed to a single location (a single 1-D scan) of a tissue site or a plurality of locations (a plurality of 1-D scans) of an area of a tissue site to generate backscattered and/or reflected radiation, where the tissue site is maintained at a desired temperature so that a temperature variation during scanning is sufficient to improve an accuracy of a calculated glucose concentration, generally the temperature variation is less than or equal to 1° C., and if a plurality of scans are collected, each scan location is separated by a distance between any two locations is between 500 nm and 20 mm. A second portion of the radiation is directed to a reflector to generate reference radiation. The backscattered and/or reflected radiation and the reference radiation are then combined and detected to produce optical coherence tomography signals. A glucose concentration is then calculated using an OCT slope or an OCT composite slope of the optical coherence tomography signals, where if multiple scans, then the number of signals (1-D scans) is sufficient to improve the signal-to-noise ratio of a composite OCT signal improving the OCT derived glucose concentration.

2. Description of the Related Art

In both diabetic and non-diabetic patients, hyperglycemia and insulin resistance commonly complicate critical illness [1-5]. In critically ill patients, even moderate hyperglycemia contributes to complications [4-8]. In diabetic patients with acute myocardial infarction, maintenance of blood glucose concentration ([Glu_{b}])<215 mg/dL (11.9 mmol/L) improved mortality at one year and 3.5 years [9-11].

In a recent clinical trial of human growth hormone to reduce catabolism in critically ill patients, mortality was doubled in the treatment group [12], perhaps because of growth-hormone induced hyperglycemia [13]. In 1548 patients (87% of whom were non-diabetic) randomized to receive conventional management or intensive insulin therapy to tightly control [Glu_{b}] between 80 and 110 mg/dL, intensive insulin therapy reduced mortality by more than 40% (from 8.0% to 4.6%) but carried a 5.0% risk of inducing severe hypoglycemia ([Glu_{b}]<40 mg/dL) [13]. Therefore, in critically ill patients, continuous glucose monitoring, ideally noninvasive, would be invaluable to guide insulin infusion to both control hyperglycemia and avoid hypoglycemia. However, no suitable noninvasive device is available.

U.S. Pat. No. 6,725,073 issued Apr. 20, 2004 disclosed a methods for measuring analyte concentration within a tissue using optical coherence tomography (OCT), incorporated therein by reference here and as set forth comprehensively below. Radiation is generated, and a first portion of the radiation is directed to the tissue to generate backscattered radiation. A second portion of the radiation is directed to a reflector to generate reference radiation. The backscattered radiation and the reference radiation is detected to produce an interference signal. The analyte concentration is calculated using the interference signal. This patent of two of the inventors set forth the basic principles of OCT and the reader is directed thereto for additional details of the OCT system. However, the method of U.S. Pat. No. 6,725,073 has not readily amenable to continuous monitoring and monitoring with temperature and/or pressure control for high accuracy.

More recently, it have been discovered that temperature variation is a tissue site undergoing OCT glucose concentration monitoring can adversely affect the OCT glucose concentration making long-term or continuous OCT glucose concentration monitoring problematic.

Thus there is a need in the art for a noninvasive reliable method and system of continuously monitoring glucose concentration in patients in order to control glucose concentration so as not to induce hyperglycemia or hypoglycemia, especially in critically ill patients that is not subject to tissue temperature fluctuations and to OCT systems that operated an elevated and maintained temperature and at a minimal and constant pressure to improve OCT glucose concentration measurement accuracy and reproducibility. This method and system is necessary for diabetics also.

### SUMMARY OF THE INVENTION

The present invention also provides a method for continuous noninvasive glucose monitoring in an animal including an human using an OCT based glucose monitoring system, where the tissue site is maintained at a constant temperature or where a temperature variation in the tissue site is less than an amount sufficient to improve an accuracy of the calculated OCT glucose concentration, generally temperature variation is less than or equal to 1° C. and/or a pressure exerted on the site is minimal and constant, generally, less than or equal to 0.1 kPa. In certain embodiment, the minimal pressure is less than or equal to 0.01 kPa. The method includes the step of generating radiation. A first portion of radiation is directed to a plurality of locations (a plurality of 1-D scans) of the tissue site maintained at a desired temperature to generate backscattered and/or reflected radiation. A second portion of the radiation is directed to a reflector to generate reference radiation. The backscattered and/or reflected radiation and the reference radiation are then detected to produce optical coherence tomography signals. A glucose concentration is then calculated on a continuous basis or periodic basis using a composite slope of the optical coherence tomography signals, where the number of signals is sufficient to improve the signal-to-noise ratio of a composite OCT signal improving the OCT derived glucose concentration. In certain embodiments, the method is directed to 1-D scans of a tissue site that does not have inhomogeneities over the area in which the 1-D scans are taken. In certain embodiments, the plurality of 1-D scans are directed over a tissue are having an area between about 200 μm×200 μm and about 2000 μm×2000 μm. In other embodiments, a distance between any pair of 1-D scans is between about 500 nm and 20 mm. In other embodiments, the distance between any pair of 1-D scans is between 1 μm and 10 mm. In certain embodiments, the area is chosen such that tissue structures having OCT characteristics that permit reliable and reproducible glucose concentration measurements. Some of the tissue characteristics that give rise to such “stable” OCT glucose measurements are continuous and/or contiguous layers, morphological properties, a degree of vascularization of the tissue or layers therein, analyte transport properties, etc. In certain embodiments, the tissue site is warmed to a desired elevated temperature and held constant at the temperature with a temperature variation of less than or equal to 1° C. The inventors have also found that besides the slope of the OCT signal other properties or parameters of the OCT signal can be used for glucose monitoring such as magnitudes of the OCT signal at certain depths, at least one depth, magnitudes of OCT signals at different depths, and/or ratio of OCT signals at at least two different depths.

The present invention also provides a method for continuous noninvasive glucose monitoring in an animal including an human using an OCT based glucose monitoring system. The method includes the step of generating radiation. A first portion of radiation is directed onto a single site of a tissue site or an area of a tissue site to generate backscattered and/or reflected radiation, where the tissue site is maintained at a desired temperature with a temperature variation of less than or equal to 1° C. during the OCT scan. A second portion of the radiation is directed to a reflector to generate reference radiation. The backscattered and/or reflected radiation and the reference radiation are then combined and forwarded to a detected and detected to produce optical coherence tomography signals. A glucose concentration is then calculated on a continuous basis or periodic basis using a single OCT slope or a composite OCT slope of the optical coherence tomography signals over the surface, where the number of signals is sufficient to improve the signal-to-noise ratio of a composite OCT signal improving the OCT derived glucose concentration. The method can also include the step of using glucose concentration values obtained from invasive samplings of blood (routinely used in critically ill patients) to calibrate the OCT-based sensor and improve OCT glucose concentration accuracy. The method is especially well suited for patients undergoing cardiac surgery, where careful control of glucose level leads to a substantial reduction in mortality and morbidity of in such patients. In certain embodiments, the tissue site is warmed to a desired elevated temperature and held constant at the temperature with a temperature variation of less than or equal to 1° C.

The present invention also provides a method for continuous noninvasive glucose monitoring in critically ill patients. The method includes the step of generating radiation. A first portion of radiation is directed to a single location of a mucosa or a plurality of locations of a mucosa such as an oral mucosa of the patient to generate backscattered and/or reflected radiation, where the tissue site is maintained at a desired temperature with a temperature variation of less than or equal to 1° C. during the OCT scan. A second portion of the radiation is directed to a reflector to generate reference radiation. The backscattered and/or reflected radiation and the reference radiation are then detected to produce optical coherence tomography signals. A glucose concentration is then calculated on a continuous basis or periodic basis using a single OCT slope or a composite slope of the optical coherence tomography signals, where the number of signals is sufficient to improve the signal-to-noise ratio of a composite OCT signal improving the OCT derived glucose concentration. The method can also include the step of using glucose concentration values obtained from invasive samplings of blood (routinely used in critically ill patients) to calibrate the OCT-based sensor and improve OCT glucose concentration accuracy. The method is especially well suited for patients undergoing cardiac surgery, where careful control of glucose level leads to a substantial reduction in mortality and morbidity of in such patients. The inventors believe that probing of mucosa may provide more accurate glucose monitoring due to better blood perfusion and glucose transport compared in the mucosa as compared to skin tissue. In certain embodiments, the tissue site is warmed to a desired elevated temperature and held constant at the temperature with a temperature variation of less than or equal to 1° C.

The present invention provides an OCT system including a light source, a optical subsystem adapted to produce a reference beam and a sample beam. The optical subsystem is also configured to direct the sample beam onto a plurality of sites of a tissue or to direct the sample beam over an area of a tissue producing a plurality of 1-D OCT scans on a continuous basis or periodic basis. The optical subsystem also includes an interferometer for combining the reference beam and a backscattered beams from each sample scan and directing the combined beams to a photodetector adapted to collect plurality of combined beams and produce a plurality of OCT signals which are then transferred to an analyzer as they are collected, where the tissue site is maintained at a desired temperature with a temperature variation of less than or equal to 1° C. during the OCT scan. The analyzer is designed to accumulate the plurality of 1-D scans and produce a composite OCT signal with improved signal-to-noise ratio and to produce a slope of the OCT composite signal and to derive a corresponding OCT glucose concentration. The analyzer can also be designed to receive invasive blood glucose data taken during the continuous monitoring time to improve OCT software calibration and signal registration. In certain embodiments, the tissue site is warmed to a desired elevated temperature and held constant at the temperature with a temperature variation of less than or equal to 1° C.

The present invention provides a computer readable media containing program instructions for measuring glucose concentration of a plurality of 1-D scan of a tissue area. The computer readable media including instructions for storing a plurality of 1-D optical coherence tomography (OCT) signals in memory. The computer readable media also includes instructions for combining the signals into a composite signal with an improved signal-to-noise ratio. The computer readable media also includes instructions for determining the glucose concentration using the composite signal. The instructions for determining the glucose concentration include determining a slope of the composite OCT signal and determining an OCT glucose concentration using the slope. The computer readable media can also include instructions to identify structures within the tissue area at a given depth in the tissue which improve the OCT glucose concentration value relative to the actual blood glucose concentration. The computer readable media also includes instructions for maintaining a temperature of the tissue site at a desired temperature with no more than a 1° C. temperature variation during the scanning. The computer readable media can also include instructions for data filtering and/or smoothing of the OCT data to improve an accuracy of OCT glucose concentration measurements and to improve a correlation between [Glu_{OCT}] and [Glu_{b}].

The present invention provides a computer readable media containing program instructions for continuously measuring glucose concentration of a plurality of 1-D scan of a tissue area. The computer readable media includes instructions for storing a plurality of 1-D optical coherence tomography (OCT) signals in memory, instruction of forming a composite OCT signal from the plurality of 1-D scans and instructions for determining the glucose concentration within the tissue using the composite signal. The instructions for determining the glucose concentration include instructions for correlating a change in the slope with an optical or morphological change in the tissue. The computer readable media can also include instructions to identify structures within the tissue area at a given depth in the tissue which improve the OCT glucose concentration value relative to the actual blood glucose concentration in the tissue. The computer readable media also includes instructions for maintaining a temperature of the tissue site at a desired temperature with no more than a 1° C. temperature variation during the scanning. The computer readable media can also include instructions for warming a tissue site and maintaining a temperature of the tissue site at a desired temperature with no more than a 1° C. temperature variation during the scanning. The computer readable media can also include instructions for data filtering and/or smoothing of the OCT data to improve an accuracy of OCT glucose concentration measurements and to improve a correlation between [Glu_{OCT}] and [Glu_{b}].

Besides deriving reliable, continuous glucose concentration values from the slope of the backscattering signal across the entire depth of tissue scanned in a 1-D scan, reliable and continuous glucose concentration also is derivable from other information contained in the backscatter signal. Reliable glucose concentrations can be derived from portion of the signal or from a collection of binned signal data. In scan including a plurality of 1-D scans, the glucose concentration can be derived from randomly or pattern selected 1-D scan or portions thereof, randomly or pattern selected 1-D scans or portions thereof, or any other combination of signal data derived from the plurality of 1D scans. The computer readable media also includes instructions for maintaining a temperature of the tissue site at a desired temperature with no more than a 1° C. temperature variation during the scanning. The computer readable media can also include instructions for warming a tissue site and maintaining a temperature of the tissue site at a desired temperature with no more than a 1° C. temperature variation during the scanning.

The present invention also provides methods for scanning a tissue site including the step of directly an OCT sample beam onto a plurality of locations of an area of a tissue so that each OCT signal is an in-depth scan of the location, a so-called A-scan. The plurality of locations can include a random collection(s) of individual locations within the area. The plurality of locations can include a patterned selection of individual locations within the area. The plurality of locations can include a random selection of contiguous subareas. The plurality of locations can include a patterned selection of contiguous subareas. The plurality of locations can include the entire area. Thus, an A-scan method collects in-depth 1-D scans at a plurality of locations within the tissue area, where the mirror in the reference beam path is moved to change the sample beam depth, i.e., an entire depth profile is scanned at each location. In certain embodiments, the tissue site is warmed to a desired elevated temperature and held constant at the temperature with a temperature variation of less than or equal to 1° C.

The present invention also provides methods for scanning a tissue site including the step of directly an OCT sample beam onto a plurality of locations of an area of a tissue so that each OCT signal is scanned at a given depth at each location, a so-called C-scan. The plurality of locations can include a random collection(s) of individual locations within the area. The plurality of locations can include a patterned selection of individual locations within the area. The plurality of locations can include a random selection of contiguous subareas. The plurality of locations can include a patterned selection of contiguous subareas. The plurality of locations can include the entire area. Thus, a C-scan method collects single depth 1-D or 2-D scans at a plurality of locations within the tissue area, where the mirror in the reference beam path is fixed at a given tissue depth. In certain embodiments, the tissue site is warmed to a desired elevated temperature and held constant at the temperature with a temperature variation of less than or equal to 1° C.

The present invention also provides methods for scanning a tissue site including the step of directly an OCT sample beam onto a plurality of locations of an area of a tissue so that each OCT signal is simultaneously depth and laterally varied. The plurality of locations can include a random collection(s) of individual locations within the area. The plurality of locations can include a patterned selection of individual locations within the area. The plurality of locations can include a random selection of contiguous subareas. The plurality of locations can include a patterned selection of contiguous subareas. The plurality of locations can include the entire area. Thus, the new scan method collects scans at a plurality of locations within the tissue area at varying depth and locations by simultaneously moving the beam over the surface to adjust the location and moving the mirror to adjust the signal depth being scanned. In certain embodiments, the tissue site is warmed to a desired elevated temperature and held constant at the temperature with a temperature variation of less than or equal to 1° C.

Regardless of the method of scanning, the methods will ultimately convert to a single OCT composite glucose concentration value. Again the size of the plurality of locations is sufficient to produce a composite signal (averaged, binned-averaged, etc.) that has improved signal-to-noise ratio and/or improved sensitivity. Regardless of the method, the system includes an apparatus for heating a tissue site and maintaining the tissue site at a constant temperature so that the temperature of the site undergoes no more that a 1° C. temperature variation.

The area to be scanned can be a regular area or an irregular area. The regular area are generally geometrical areas such as polygonal areas such as triangular areas, quadrilateral areas, pentagonal areas, hexagonal areas, etc. or circular or oval areas.

The present invention also provides multi-wavelength OCT, where one or more wavelengths (single wavelength or narrowly banded wavelength-narrow wavelength bandwidth) are used in OCT scanning. The scanning method can include performing a first 1-D scan at a location at a first frequency and then a second 1-D scan at the same location at a second frequency. The method can include making additional 1-D scans at other frequencies as well, but generally the inventors believe that two wavelength are sufficient if judiciously selected. Alternatively, the method can include scanning a portion or all of a tissue area at a first wavelength and then scanning the same or different portion or all of the tissue area with a second wavelength. The wavelength are selected from the electromagnetic spectrum between about 700 and about 2000 nm. In certain embodiments, the first wavelength is a longer wavelength generally between about 1300 nm and about 2000 nm and the second wavelength is a shorter wavelength generally between about 700 nm and 1300 nm. The longer wavelength data correlates with water contributions to the OCT signal and the longer wavelength data is thus used to correct the OCT data at shorter wavelength, which generally correlates between glucose contributions to the OCT signal. The longer wavelength OCT signals are more water specific allowing efficient removal of water contributions, while shorter wavelength improve contrast. The combination of the two signal types can be used to enhance glucose specificity by better accounting for artifacts do to water. Alternatively, the OCT scan can be collected at one or more glucose specific wavelengths, but currently no light source are commercially available that generate light at those wavelengths. The two wavelength specific signals can be combined using an acceptable mathematical technique such as ratiometric analysis. In certain embodiments, the tissue site is warmed to a desired elevated temperature and held constant at the temperature with a temperature variation of less than or equal to 1° C.

### BRIEF DESCRIPTION OF THE DRAWINGS

The invention can be better understood with reference to the following detailed description together with the appended illustrative drawings in which like elements are numbered the same.

FIGS. 1A & B depict plots showing a slope of OCT signals obtained from rabbit skin and corresponding skin temperature and blood glucose concentration versus time during: (A) skin heating resulted in changes in skin temperature; and (B) glucose clamping experiments with relatively stable skin temperature.

FIG. 2A depicts a schematic diagram of an embodiment of an Optical Coherence Tomography (OCT) system of this invention.

FIG. 2B depicts a schematic diagram of a fiber optics embodiment of an Optical Coherence Tomography (OCT) system of this invention.

FIGS. 3A & B depict a holder with heating element for the OCT probe.

FIG. 4 depicts a temperature and corresponding voltage vs. time during in vivo glucose monitoring experiment in a pig.

FIG. 5A depicts OCT signal slope and blood glucose concentration vs. time, where the experiment was performed without temperature control.

FIG. 5B depicts OCT signal slope and blood glucose concentration vs. time, where the experiment was performed with the temperature control.

FIG. 6 depicts temperature and corresponding voltage vs. time in another pig.

FIG. 7 depicts OCT signal slope and blood glucose concentration vs. time in another animal, where the experiment was performed with the temperature control.

FIG. 8 depicts a plot of the slope of OCT signals recorded from a human subject and blood glucose concentration measured at different time during oral glucose tolerance test (OGTT). Blood glucose concentration was measured every 15 minutes.

FIG. 9 depicts a plot of the slope of OCT signals recorded from a human subject and blood glucose concentration measured at different time during OGTT. Blood glucose concentration was measured every 5 minutes.

FIG. 10A depicts a plot of the slope of OCT signals obtained from rabbit ear during glucose clamping experiment with scanning over 0.2 mm×0.2 mm area.

FIG. 10B depicts a plot of the slope of OCT signals obtained from the rabbit ear with scanning over 0.2×0.2 mm area vs. blood glucose concentration.

FIG. 11A depicts an OCT image of pig skin; 1 and 2 show the layers in which the correlation coefficients between the OCT signal slope and blood glucose concentration were highest.

FIG. 11B depicts a corresponding histological section of pig skin; 1 and 2 demonstrate the layers at which the correlation coefficients between the OCT signal slope and blood glucose concentration were highest.

FIG. 12 depicts changes in OCT signal slope with varying pressure and temperature are plotted with no glucose injection in a pig: glucose concentration [Glu_{b}] (circles) was constant.

FIG. 13A depicts an embodiment of a weight compensated OCT probe of this invention.

FIG. 13B depicts an embodiment of a weight compensated OCT probe of this invention.

FIG. 14 depicts a filtered OCT signal slope vs. [Glu_{b}] in a pig when temperature control was used and no pressure was applied to skin.

FIG. 15A depicts OCT signal slope and blood glucose concentration measured during oral glucose tolerance test (OGTT) in a healthy volunteer. 1—temperature control is off; 2—temperature control is on, but temperature is not in the range of 39.0° C.±0.3° C.; 3—temperature is stabilized in the range 39.0° C.±0.3° C.; 4—temperature control is off. Initial temperature: 32.7° C.; final temperature: 33.8° C.

FIG. 15B depicts a FOURIER filtering of the OCT signal slope (filtered) measured during oral glucose tolerance test (OGTT) in a healthy volunteer.

FIG. 15C depicts relative changes in the OCT signal slope (dots) during the increase of blood glucose concentration. The linear regression (line) was used for [Glu_{OCT}] calculation.

FIG. 15D depicts differences between [Glu_{OCT}] and [Glu_{b}] during decrease of blood glucose concentration vs. [Glu_{b}].

### DETAILED DESCRIPTION OF THE INVENTION

The inventors have developed a novel optical coherence tomography (OCT) technique for noninvasive, continuous glucose monitoring based on interferometric measurement and analysis of low-coherent light backscattered from specific layers of tissues under temperature controlled conditions. The inventors demonstrated that the accuracy and reproducibility of noninvasive glucose monitoring is dependent on tissue temperature. The inventors have shown that temperature variation of less than 1° C. do not worsen accuracy of glucose monitoring, but temperature variation of more than 1° C. results in changes of the OCT signal. The inventors have demonstrated that temperature variations of more that 1° C. Substantially worsen accuracy of glucose monitoring in animals including humans and, therefore, may substantially worsen accuracy of glucose monitoring in non-diabetic and diabetic patients. The inventors have found that tissue temperature control can be used to minimize adverse temperature affects on OCT glucose concentration derived values and to improve the accuracy and reproducibility of glucose monitoring with OCT. The inventors have found that an improved method for OCT blood glucose concentration monitoring using low-coherence interferometry (LCI) can be implemented by performing OCT measures of tissues under tissue temperature control. The method includes the step of warming the tissue to a desired temperature to provide better blood perfusion to the probed tissue, to decrease temperature fluctuations in the tissue during OCT measuring and to improve glucose transport through the tissue being monitored either using an OCT system and probe or a LCI system and probe.

The present invention is designed to use temperature control of tissue during OCT scanning on a single scan, intermittent scan, periodic scan or continuous scan basis. In certain embodiment, the temperature control OCT apparatus simply maintains the temperature at a constant temperature during OCT scans to maintain a temperature variation in the tissue to of less than or equal to 1° C. In other embodiments, the temperature control during OCT scans on a single scan, intermittent scan, periodic scan or continuous scan basis, where the temperature control includes warming the tissue to an elevated temperature and maintaining the temperature so that a temperature variation in the tissue to of less than or equal to 1° C. In other embodiments, the temperature control during OCT scans on a single scan, intermittent scan, periodic scan or continuous scan basis, where the temperature control includes cooling the tissue to a lowered temperature and maintaining the temperature so that a temperature variation in the tissue to of less than or equal to 1° C. Temperature controlled OCT glucose measuring is especially well suited for many patients and normal subjects including, but not limited to: diabetic patients, critically ill (both diabetic and non-diabetic) patients, surgical (both diabetic and non-diabetic) patients, hospital (both diabetic and non-diabetic) patients. The inventors have designed and built a system for temperature control and performed glucose monitoring experiments in vivo with the system. The results of our studies demonstrate that if tissue temperature control is used, the glucose monitoring, in particular, long-term (for more than about half an hour) glucose monitoring has substantially higher accuracy and reproducibility with clinically acceptable lag time of about 2.5 min.

The inventors have demonstrated that the accuracy and reproducibility of noninvasive glucose monitoring is dependent on tissue temperature and showed that temperature variation of less than 1° C. do not worsen accuracy of glucose monitoring (in animal and clinical studies), but temperature variation of more than 1° C. substantially worsen accuracy of glucose monitoring in animals potentially making OCT glucose monitoring in non-diabetic subjects and diabetic patients to be problematic. The inventors then developed a temperature control system for OCT system and used it in studies in vivo. The inventors then demonstrated that if tissue temperature control is used, the glucose monitoring, in particular, long-term (for more than about half an hour) glucose monitoring has substantially higher accuracy and reproducibility and that low-coherence interferometry with tissue temperature control is an effective system of long-term glucose monitoring.

The inventors also developed tissue warming as a technique to provide better blood perfusion to the probed tissue and therefore, better glucose transport to the probed area. Controlled, elevated temperature OCT techniques are well suited for two groups of patients: diabetic patients and critically ill patients (both diabetic and non-diabetic).

This invention is not obvious to a person having ordinary skill in the art to which this invention pertains, because OCT signal slope was not known to be dependent on tissue temperature until this invention. The inventors demonstrated that controlled temperature OCT, controlled elevated temperature OCT and controlled lower temperature OCT technologies are capable of glucose monitoring in phantoms and in vivo in animals and humans. The inventors demonstrated that temperature variations of more that 1° C. substantially worsen accuracy of glucose monitoring in animals and, therefore, may substantially worsen accuracy of glucose monitoring in non-diabetic subjects and diabetic patients. The inventors developed a temperature control system and used it in studies in vivo. The inventors demonstrated that if tissue temperature control is used, the glucose monitoring, in particular, long-term (for more than about half an hour) glucose monitoring has substantially higher accuracy and reproducibility.

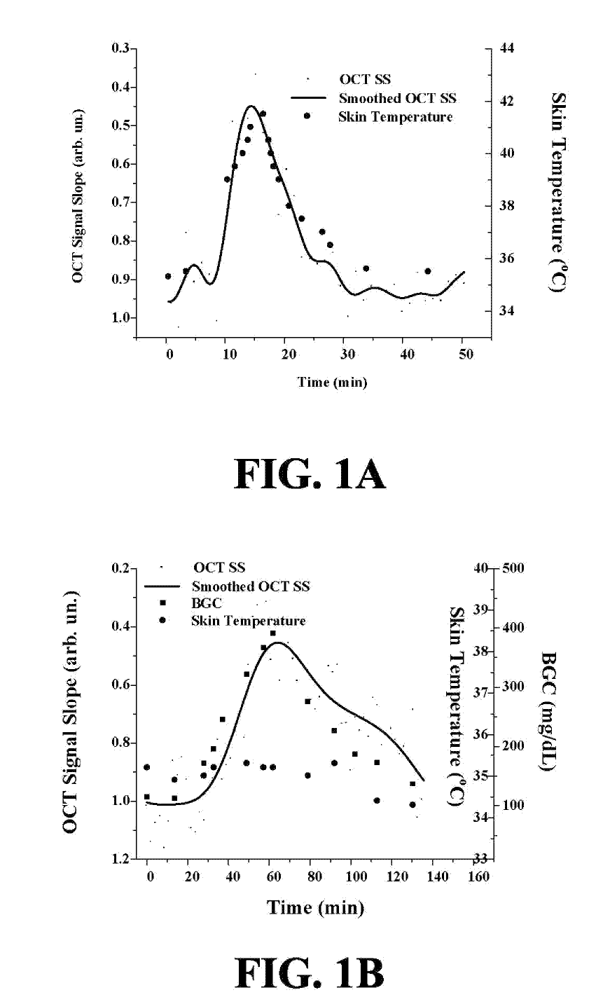

Variation of temperature may produce changes in the OCT signal slope. Several experiments were performed to demonstrate the effect of skin temperature on the OCT signal slope. Experiments performed in skin tissue in vivo showed a dependence of the OCT signal slope on the skin temperature. FIG. 1A shows a typical result obtained from rabbit skin during skin heating with hot air produced by a heat gun. Skin temperature was monitored using a thermocouple placed on the skin surface near the OCT probe. The results show decrease of the OCT signal slope with increase of skin temperature from 35° C. to 42° C. and increase of the OCT signal slope back to the original values due to passive tissue cooling. Therefore, tissue heating change tissue properties and adverse affects OCT signal slope.

Minor temperature fluctuations of the skin (≦±1° C.) did not change the OCT signal slope in a control experiment without heating and did not adversely affect the accuracy of glucose monitoring with OCT as shown in FIG. 1B.

If this technique is used without temperature control by diabetic patients at home, or in critically ill, surgical, or hospital patients, and the tissue temperature varies, it will result in unacceptable accuracy and reproducibility of glucose monitoring—problematic results. The inventors, therefore, discovered that tissue warming and/or temperature control can be used in OCT glucose monitoring to yield OCT glucose value having high accuracy and reproducibility.

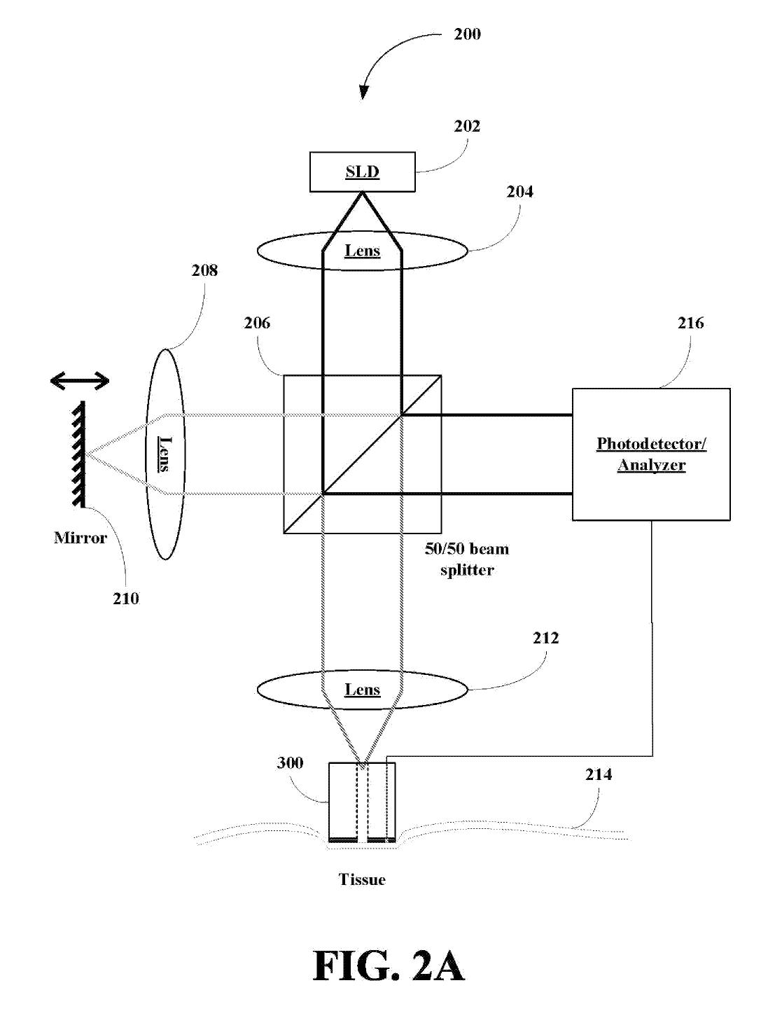

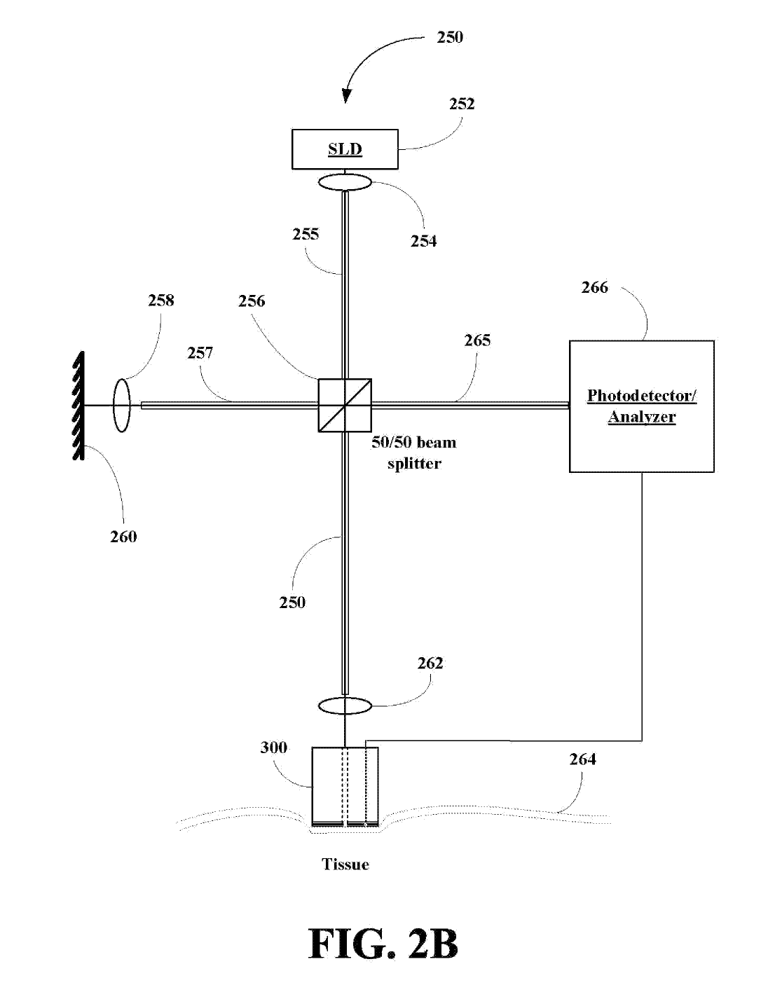

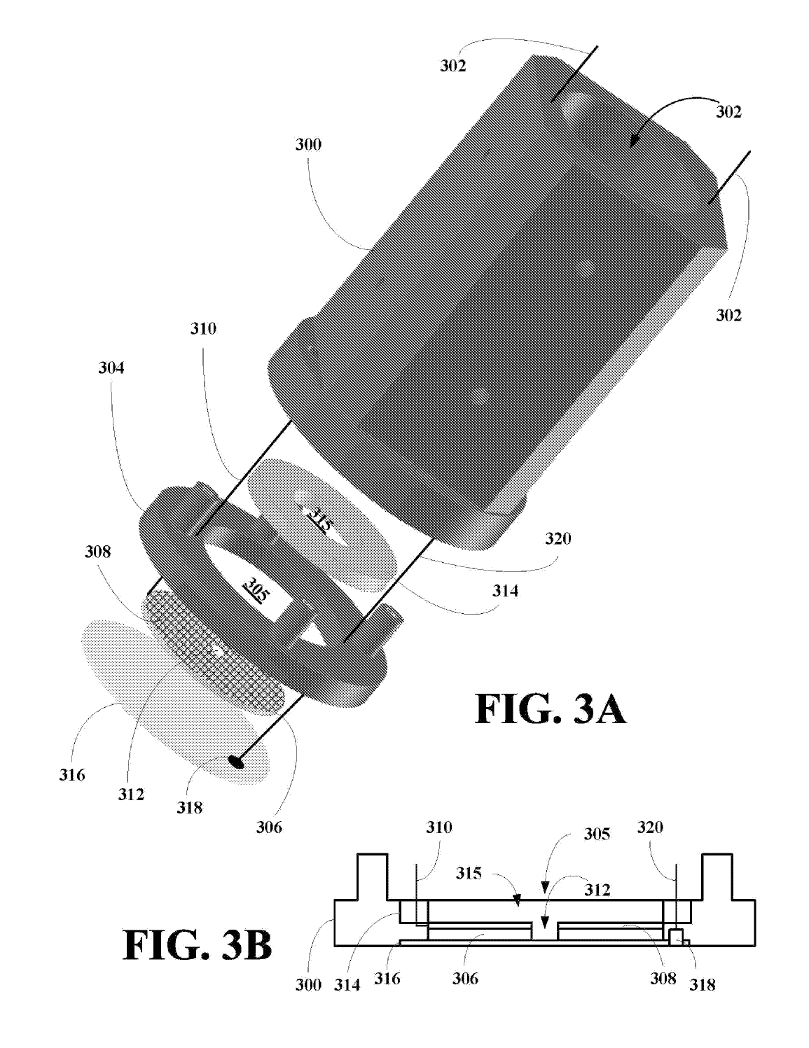

OCT is a new optical diagnostic technique that provides depth resolved images of tissues with resolution of about 10 μm or less at depths of up to 1 mm. The present invention is directed to the use of the OCT technique for monitoring of blood glucose concentration by measuring and analyzing light coherently backscattered from specific tissue layers as demonstrated in animal and clinical studies to continuously, non-invasively and accurately monitoring glucose monitoring [46-53]. The basic principle of the OCT technique is to detect backscattered photons from a tissue of interest within a coherence length of a light source using a two-beam interferometer. An OCT system for use in this invention, generally 200, is shown in FIG. 2A. Light from a superluminescent diode (SLD), a light source with low coherence, 202 passed through a first lens 204 and then directed to a 50/50 beam splitter 206. Half of the beam is directed through a second lens 208 and onto a mirror 210. The beam is reflected at the mirror 210 and reenters the beam splitter 206. A second half of the split initial beam is directed through a third lens 212 onto a tissue 214. Backscattered light is collected for the lens 212 and enters the splitter 206. The combined light is then forwarded to a photodetector/analyzer 216, where an interference between the two beams is used to calculate a slope of the OCT signal. The system 200 also includes a temperature controlled probe housing 300, explained in detail in FIG. 3. Thus, the system aims light at objects to be scanned using the sample beam existing the beam splitter. Light scattered from the tissue is combined with light returned from the reference arm, and a photodiode detects the resulting interferometric signal. Intereferometric signals can be formed only when the optical path length in the sample arm matches the reference arm length within coherence length of the source (10-15 μm). By gathering interference data at points across the surface, cross-sectional 2-D images can be formed in real time with resolution of about 10 μm at depths of up to one millimeter or deeper depending on the tissue optical properties [54-59].

Referring now to FIG. 2B, a fiber optics version of an OCT apparatus used in the examples set forth below generally, 250, is shown. Light from a superluminescent diode (SLD), a light source with low coherence, 252 can optionally passed through a first lens 254 into a first optical fiber or fiber bundle 255 and then directed to a 50/50 beam splitter 256. Half of the beam is directed into a second optical fiber or fiber bundle 257 and optionally through a second lens 258 and onto a mirror 260. The beam is reflected at the mirror 260 and into the optical fiber 257 and reenters the beam splitter 156. A second half of the split initial beam is directed into a third optical fiber or fiber 261 and optionally through a third lens 262 and then onto a tissue 264. Backscattered light is then directed into the third optical fiber 261 or optionally through the third lens 262 and into then the third optical fiber 261 and then reenters the splitter 256. A portion of the backscattering beam and the reference beam are combined by the splitter 256 and forwarded through a fourth optical fiber 265 to a photodetector/analyzer 266, where an interference between the two beams is used to calculate a slope of the OCT signal. The system 250 also includes a temperature controlled probe housing 300, explained in detail in FIG. 3.

The inventors developed a temperature control system and used it in studies in vivo. An embodiment of a probe housing 300 of the temperature controlled OCT system of this invention is shown in FIG. 3. Looking at FIG. 3, the temperature controlled probe housing 300 is shown to include a light aperture 302 and an OCT probe 304 attached with plastic screws (not shown) to the housing 300. The probe 304 having an aperture 305 therethrough and including a metal plate 306, where the metal plate is made of a metal with high heat conductivity such as Cu, Fe, Co, Ni, Zn, Ru, Rh, Pd, Ag, Cd, Os, Ir, Pt, or Au as well as alloys thereof and other high heat conductivity metals. Optionally, the metal plate 306 is heated by a heating element or wire 308 connect to a power supply such as a variable DC power supply or a DC battery via connecting wires 310, shown here disposed on a top surface of the metal plate 306 and having an aperture 312. If the probe 304 is not heated, then the aperture 312 would be through the metal plate 306. In those applications, where temperature control is needed, but heating is not, then no heating means is needed and only the heat sink or metal plate 306 is needed. It should be recognized by ordinary artisans that any heating means can be used provided that it is amenable to safely warm tissue of an animal including a human. In certain embodiments, the metal plate 306 is a copper plate having a thickness about one millimeter. The metal plate 306 include an aperture coincident with the aperture 312 of the heating element 310 in a middle of the plate 304. Both apertures having a diameter sufficient to allow the sample OCT beam to pass therethrough. Generally, the apertures have a diameter of about 2.5 mm. The probe 304 also includes a temperature isolator 314 having an aperture 315 therethrough. The temperature insulator 314 can be constructed of any thermal insulator such as a polymer, rubber, a ceramic, a gas, or the like. The insulator 314 is adapted to direct most of the heat to the skin for a better efficiency. The probe 304 can also include a transparent plate 316 glued to a bottom surface of the metal plate 306, where the transparent plate 316 is adapted to form a smooth contact surface with the tissue site to be scanned such as a site on an animal or human's skin.

The heating voltage was to the heating element 306 varied between about 3V and about 6V to provide stable temperature in different animals/subjects. The probe 302 can also include a thermocouple 318 with an accuracy of 0.2° C. adapted to measure actual tissue temperature during OCT scans. The thermocouple 306 is connected to the photodetector/analyzer 216 or 266 of an OCT systems 200 or 250, respectively, via wires 320 so that the temperature data can be recorded as a scan parameter.

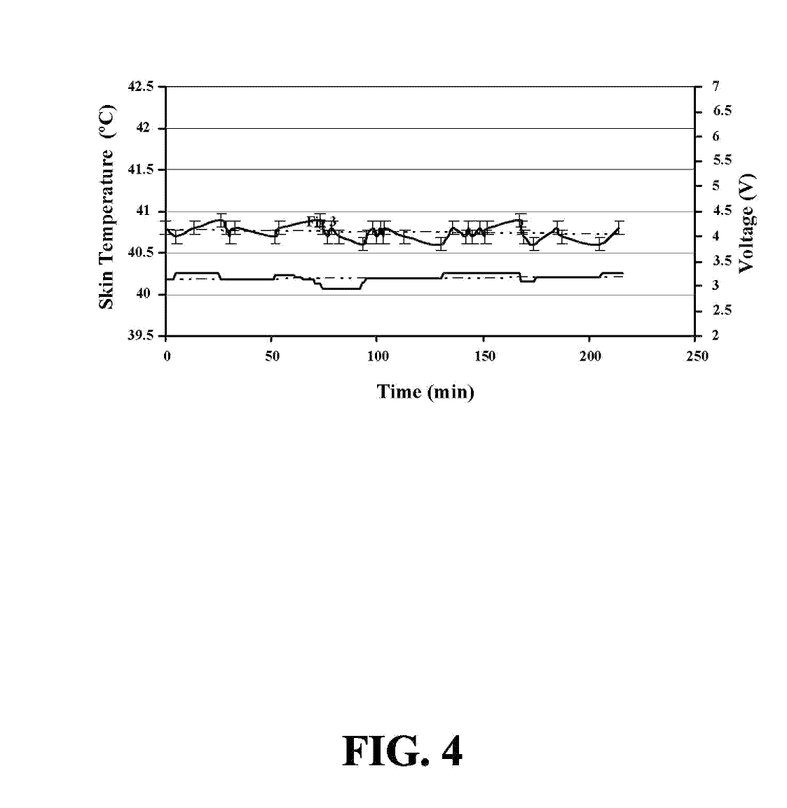

Skin temperature measured during glucose monitoring experiment is presented in FIG. 4. The figure shows also the voltage applied to the heating element. The voltage can be adjusted to provide temperature at or above normal skin temperature with the accuracy of 0.2° C.

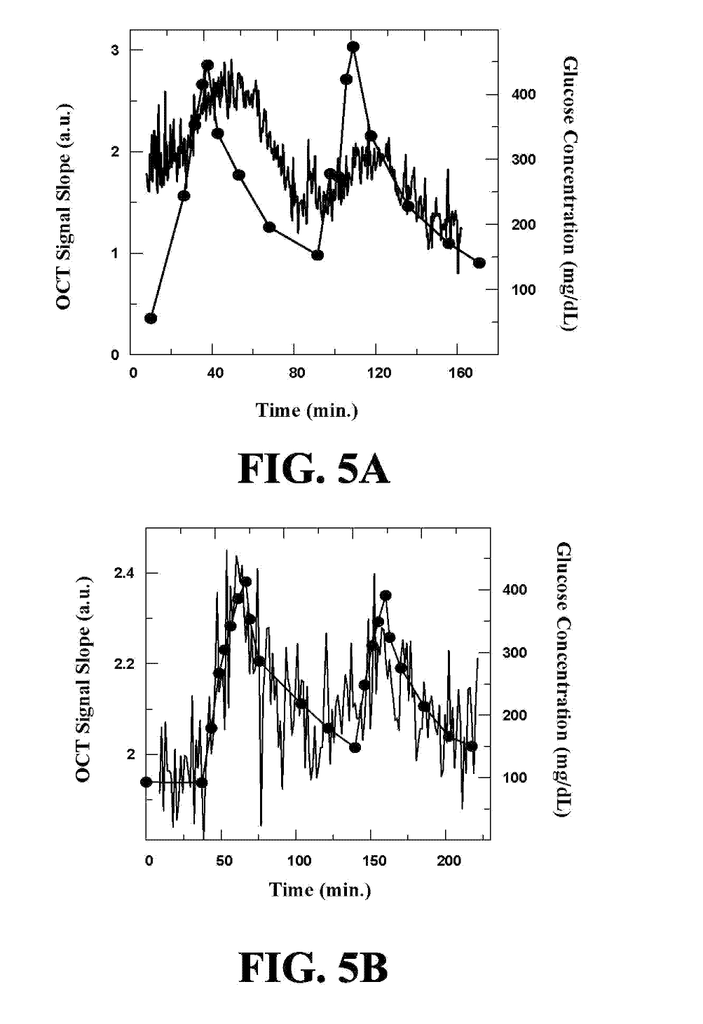

The temperate control system provided much better accuracy and reproducibility of glucose monitoring. FIG. 5A shows OCT signal slope and glucose concentration during two cycles of glucose injections when the temperature control system was not used. Due to variation of pressure and temperature OCT signal slope had long-term drift of the order of tens of minutes that is typical for changes in blood glucose concentration. Moreover, the lag time between the blood glucose concentration and OCT signal slope was approximately 20 min. The lag time varied between 0 and 40 min in different humans and animals when temperature control was not used. When the holder with the temperature control system was used as shown in FIG. 5B, OCT signal slope closely followed changes in glucose concentration with almost no lag time and the long-term drift.

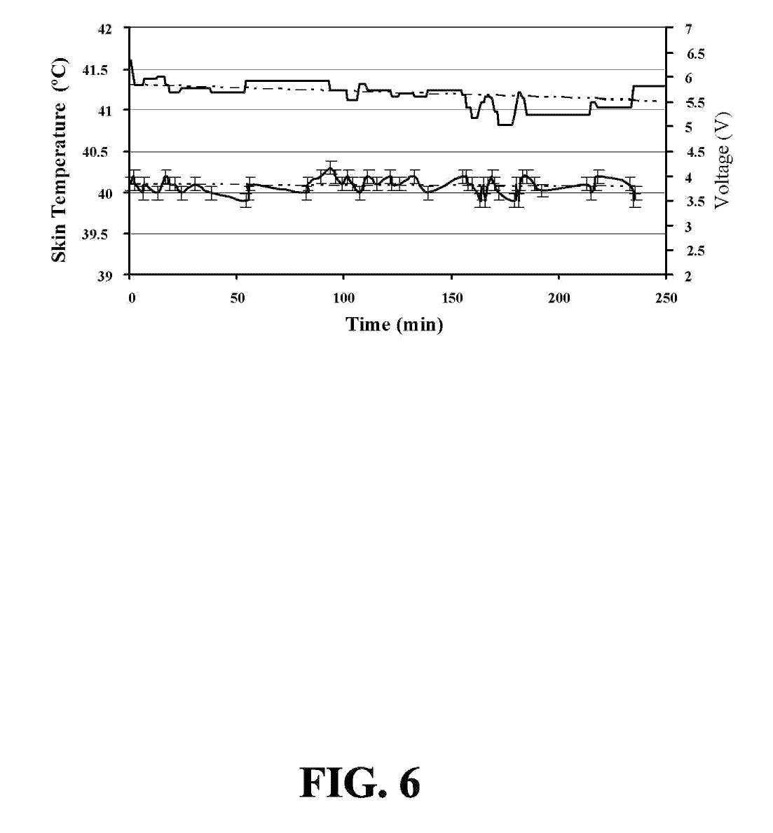

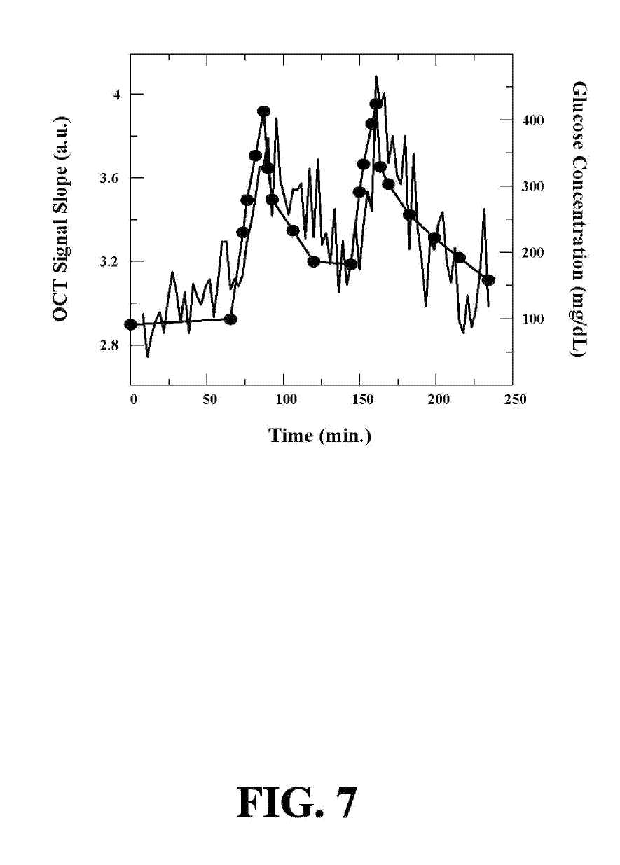

Similar results were obtained in other experiments. FIG. 6 shows skin temperature and heating voltage during glucose monitoring experiment in another pig. A higher voltage was required for this pig compared to the first one due to differences in physiology. FIG. 7 shows OCT signal slope and glucose concentration during two cycles of glucose injections in this pig. The OCT signals slope closely followed glucose concentration with minimal lag time.

The inventors have concluded that temperature control provides much better accuracy and reproducibility of glucose monitoring and reduces the lag time to attain clinically acceptable glucose level of 2.5 min on average. The inventors have demonstrated that if tissue temperature control is used, the glucose monitoring, in particular, long-term (for more than about one hour) glucose monitoring has substantially higher accuracy and reproducibility.

By averaging of the 2-D OCT images into a single 1-D composite OCT signal in depth, one can measure the optical properties of tissue or a specific tissue layer by analyzing the profile of the OCT signal. By varying the location of the 1-D composite OCT signal, a 3-D map of the tissue can be constructed with information about local profusion rate, local glucose concentration and local water concentration can be determined. The inventors have also found that certain structures within a tissue prove more reliable and reproducible OCT glucose concentration values. Thus, the method can also be used to determine those structures within a tissue or those tissues that can provide the most reliable and reproducible OCT glucose concentration values for continuous monitoring. In certain embodiments, the tissue is a mucosa, while in other embodiments the tissue structure is near a dermis-subdermis boundary and near a papillary and reticular junction in the dermis.

The inventors demonstrated that the higher resolution of OCT provides accurate and sensitive measurements of scattering from specific tissue layers. Moreover, due to coherent light detection, photons that are scattered from other tissue layers as well as diffusively scattered photons do not contribute to the OCT signal recorded from the tissue layer of interest. These features of the OCT technique provide accurate, sensitive, noninvasive, and continuous monitoring of blood glucose concentration with the proposed sensor.

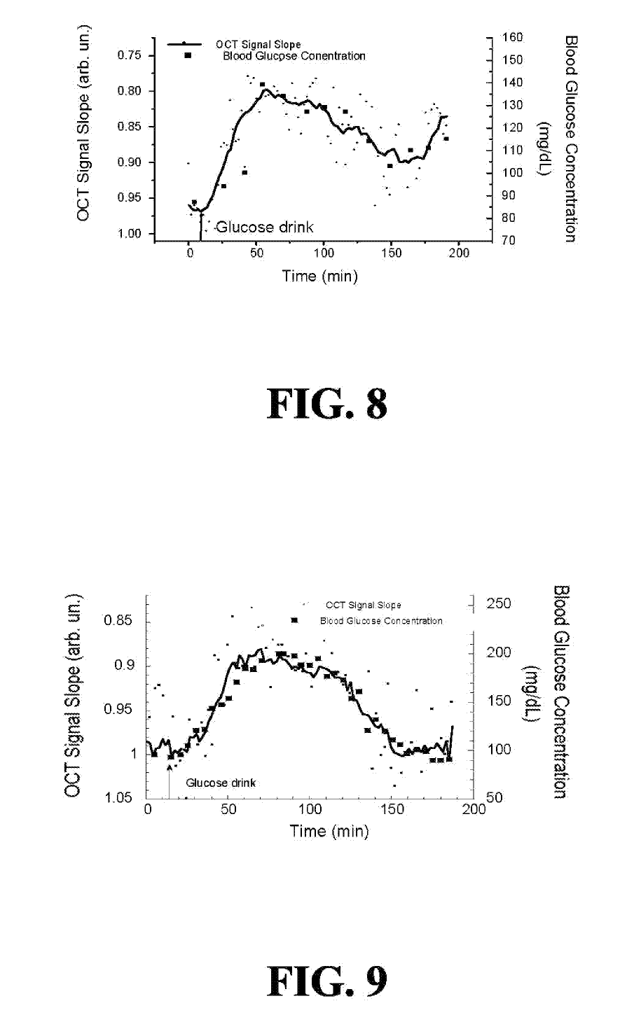

The inventors demonstrated in animal and clinical studies that the OCT technique is capable of continuous and noninvasive glucose monitoring when OCT signal slopes are measured from specific tissue layers [46-53]. Typical results obtained in clinical studies are shown in FIG. 8 and FIG. 9. The blood glucose concentration was measured each 15 and 5 minutes as shown in FIGS. 8 and 9, respectively, during the experiments. Decreases and increases of the OCT signal slope followed the changes in blood glucose concentration. The slopes were calculated at the depth of 550-600 μm as shown in FIG. 8 and 380-500 μm as shown in FIG. 9. The slopes changed significantly ˜17% with changes in glucose concentration from 90 to 140 mg/dL (first volunteer) and ˜15% with the changes in glucose concentration from 100 to 200 mg/dL (second volunteer).

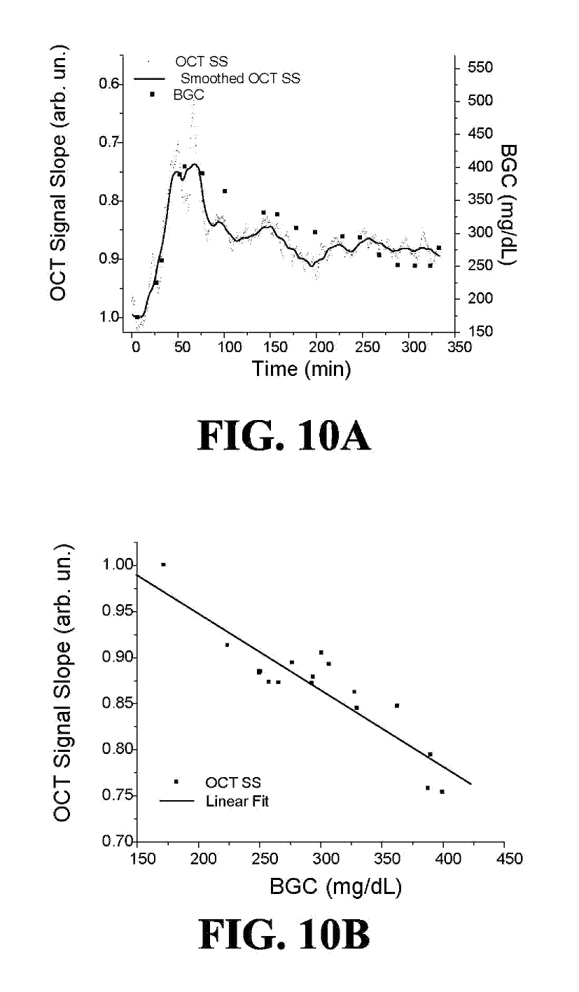

The inventors performed animal tests that included glucose clamping and square scanning of the beam over 0.2×0.2 mm (200 μm×200 μm) area of rabbit ear skin as shown in FIGS. 10A & B. Scanning over an area substantially reduced the scattering of the OCT data points compared to data obtained with linear scanning under similar conditions. Because areas have been shown experimentally improve OCT glucose measurement accuracy and precision by improving signal-to-noise ratio and other signal properties, a plurality of scans at specific locations within the area without scanning every location in the area will also give rise to improved OCT glucose measurements. Thus, the area scanning can be over the entire surface in any type of scanning pattern or the area scanning can be over patterned or randomly selected locations in the area.

The results of these studies demonstrated that 2-D lateral scanning of the incident OCT beam over an area such as a square provides better signal stability, reduces noise, and improves accuracy of the calculated glucose value. The 2-D lateral scanning can be performed over a rectangular, circular, elliptical, or any other 2-D area.

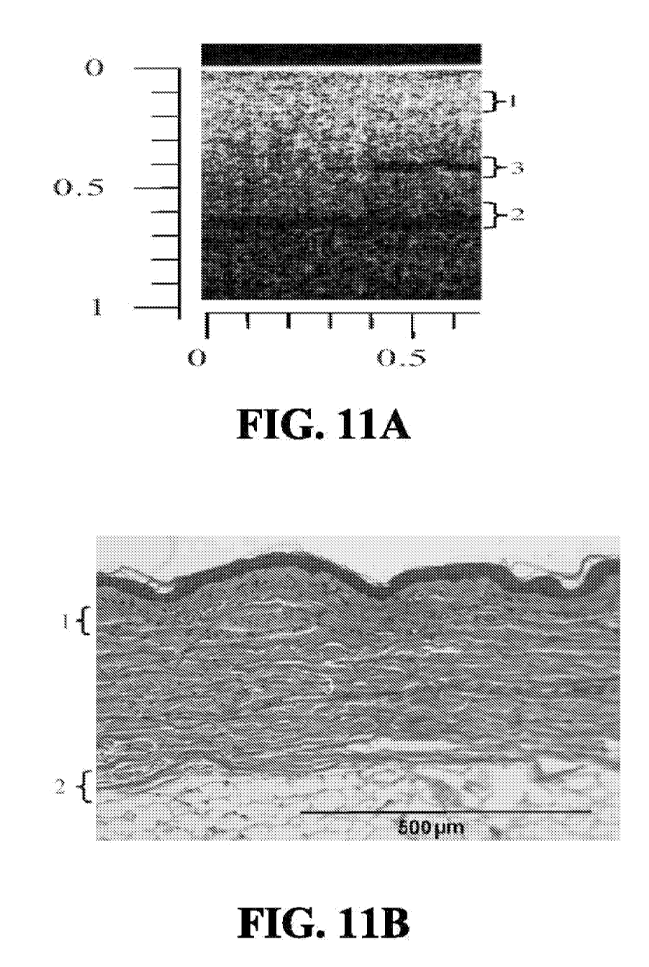

The inventors also identified specific skin layers in which an improved or best correlation between OCT signal slope and blood glucose concentration was obtained. The experiments were performed in young, 4-5 months old pigs (best model of human skin). Comparison between H&E-stained sections was performed to identify these layers on the OCT images as shown in FIGS. 11A&B that were used to map the OCT signals onto the H&E-stained section. Although the OCT signal slope correlated well with blood glucose concentration in all pigs, the best correlations between the OCT signal slope and blood glucose concentration occurred in specific depths within the tissue being imaged. A strong correlation between blood glucose concentration and OCT signal slope was found at the boundary between the dermis and subdermis. The OCT signal slope also correlated with blood glucose concentration in other skin layers, especially near the papillary and reticular junction in the dermis.

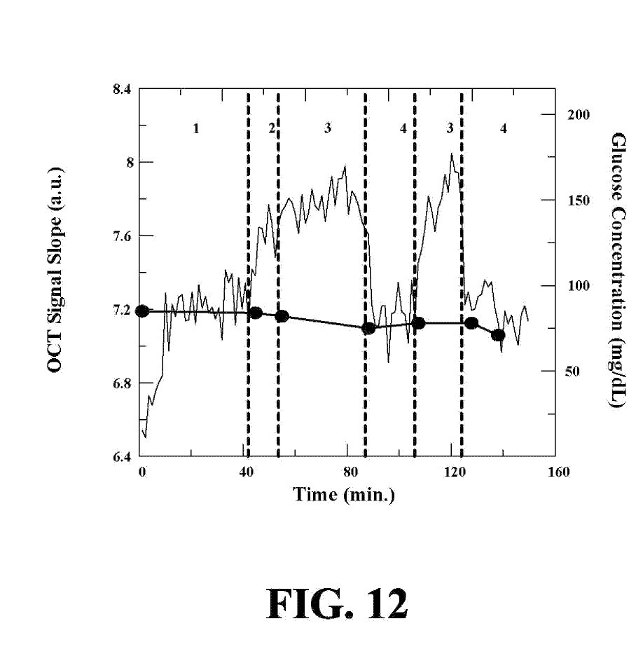

Referring now to FIG. 12, changes in OCT signal slope with varying pressure and temperature are plotted with no glucose injection in a pig: glucose concentration [Glu_{b}] (circles) was constant. The OCT data was collected using across an area as set forth above concerning obtaining a plurality of 1-D scan within a portion or an entire area of a tissue site. The data evidences that the release of pressure (2 kPa) applied by a OCT probe (weight: 400 g) results in abrupt changes in the OCT signal slope even under stable temperature conditions. These abrupt changes are apparent in area 3 (P=2 kPa) compared to area 4 (P=0 kPa). Thus, OCT scans are sensitive to both pressure and temperatures fluctuations in the tissue site being scanned. In order to obtain improved OCT data, the OCT scan are to be performed under controlled temperature and pressure conditions. The inventors have found that the pressure can be made minimal by constructing an OCT probe handle that exerts a minimal pressure on the tissue site to be scanned, but still includes the probe and temperature control components. An embodiment of such a handle is described herein. In certain embodiments of the method for scanning of this invention, the pressure is a constant minimal pressure, while the temperature is a constant elevated temperature. In other embodiments, the pressure is minimal and constant, while the temperature is between about 33° C. and 45° C. with a temperature variation of less than or equal to 1° C. In other embodiments, the pressure is minimal and constant, while the temperature is between about 37° C. and 41° C. with a temperature variation of less than or equal to 1° C. In other embodiments, the pressure is minimal and constant, while the temperature is between about 38° C. and 41° C. with a temperature variation of less than or equal to 1° C.

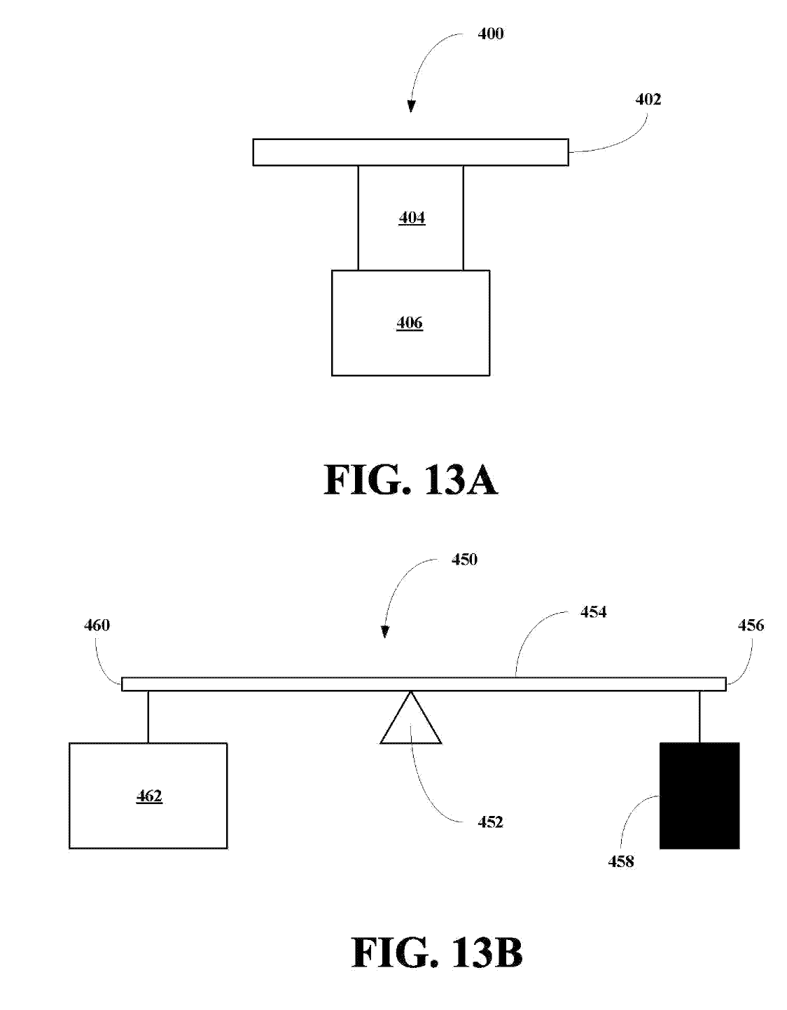

Because the inventors have found that pressure control is important for achieving high accuracy OCT glucose concentration measurement, the inventors have developed OCT probes that are adapted to maintain a constant minimal pressure of the probe on the skin surface at the site to be scanned, where the probe includes a weight compensation means. While these probes are adapted to compensate for probe weight, especially with probes that include heating and temperature control components, if the OCT probe is made sufficient light in weight, the probes will not require weight compensation means to achieve a minimal and constant pressure at the tissue site to be scanned. Referring now to FIG. 13A, an embodiment of a weight compensated OCT probe of this invention, generally, 400, is shown to include a stationary member 402, a spring or biased member 404 and an OCT probe 406. The probe 406 is in optical communication with the beam splitter of FIGS. 2A or 2B and is adapted to receive the sample beam and return the backscattered beam. The probe 406 can also include the heating and temperature control components of FIG. 3. The spring-based system 400 was designed to minimize a pressure applied by the probe to the skin surface. Referring now to FIG. 13B, another embodiment of a weight compensated OCT probe of this invention, generally, 450, is shown to include a stationary pivot point 452 and an arm 454 mounted on the stationary pivot point 452. At one end 456 of the arm 454, a weight 458 is attached and at the other end 460 an OCT probe 462 is attached. The arm 454 is moved relative to the pivot point 452, until a minimal pressure is exerted on the skin by the probe 462. While two weight compensation means are described, any weight compensation means can be used to ensure that the OCT probe exerts a minimal and constant pressure on the tissue at the site to be scanned. Alternatively, if the housing 300 is made of sufficient light weight materials, then a weight compensation system would not be needed as the probe would exert minimum pressure on the tissue site.

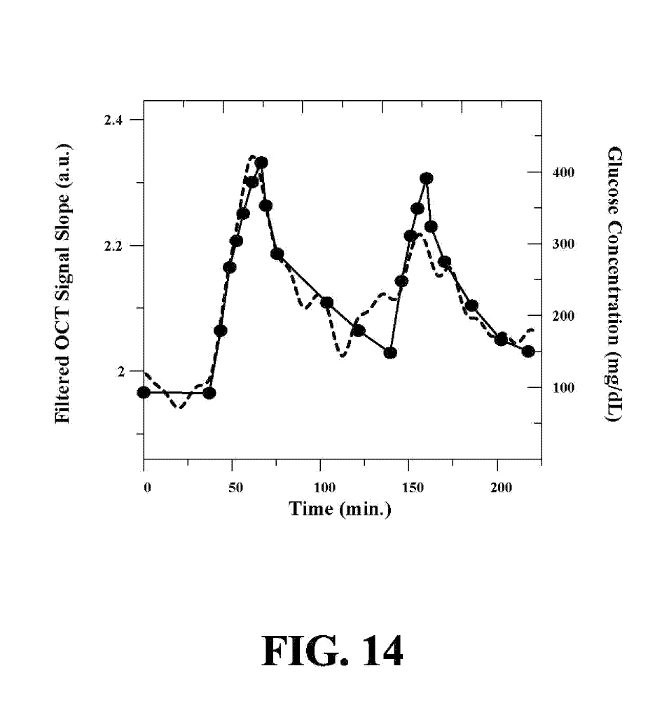

The inventors have found that temperature and pressure control used in combination with Fourier filtering provided an improved correlation of the OCT signal slope with [Glu_{b}]. FIG. 14 shows a filtered OCT signal slope vs. [Glu_{b}] in a pig when temperature control was used and no pressure was applied to skin. FIG. 14 clearly shows a more robust correlation between the OCT signal slope with [Glu_{b}]. Thus, an accuracy of the OCT glucose concentration measurement and the correlation between the [Glu_{b}] and [Glu_{OCT}] can be improved by subjecting the OCT data to a mathematical smoothing or filtering routine. For instance, Fourier filtering has been used demonstrating the improvement in an accuracy of the OCT glucose concentration measurement and the correlation between the [Glu_{b}] and [Glu_{OCT}]. Thus, filtering and/or smoothing of the OCT data such as Fourier filtering reduces noise and improves accuracy.

The inventors have discovered that OCT glucose monitoring can be improved by performing OCT scans under a combination of temperature control, pressure control, data filtering and/or area scanning. The combination of any of these factors improves an accuracy of OCT glucose concentration measurements and improves the correlation between the [Glu_{b}] and [Glu_{OCT}], while the combination of all four factors provides OCT glucose measure that are near clinically acceptable accuracy limits.

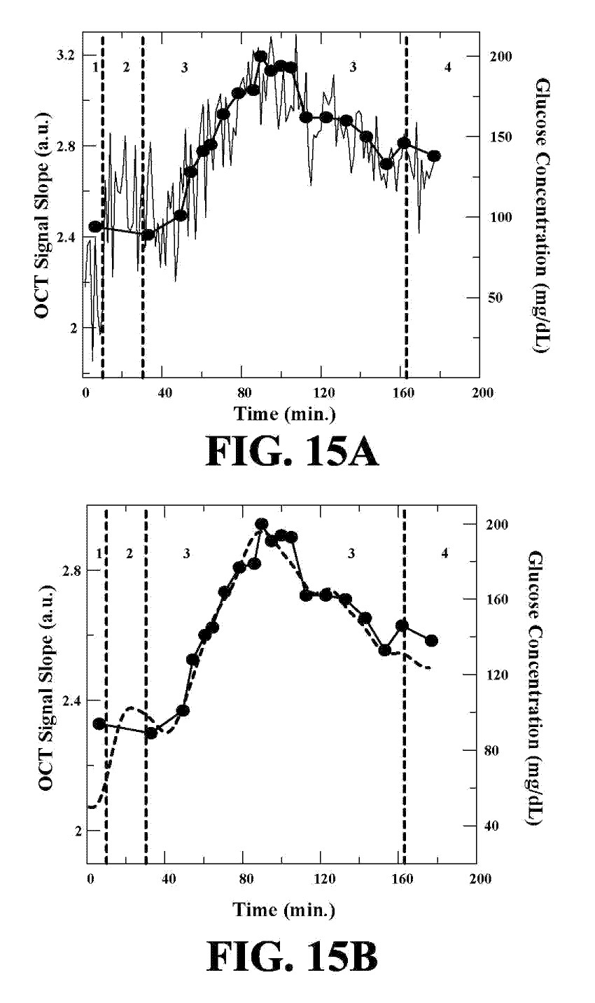

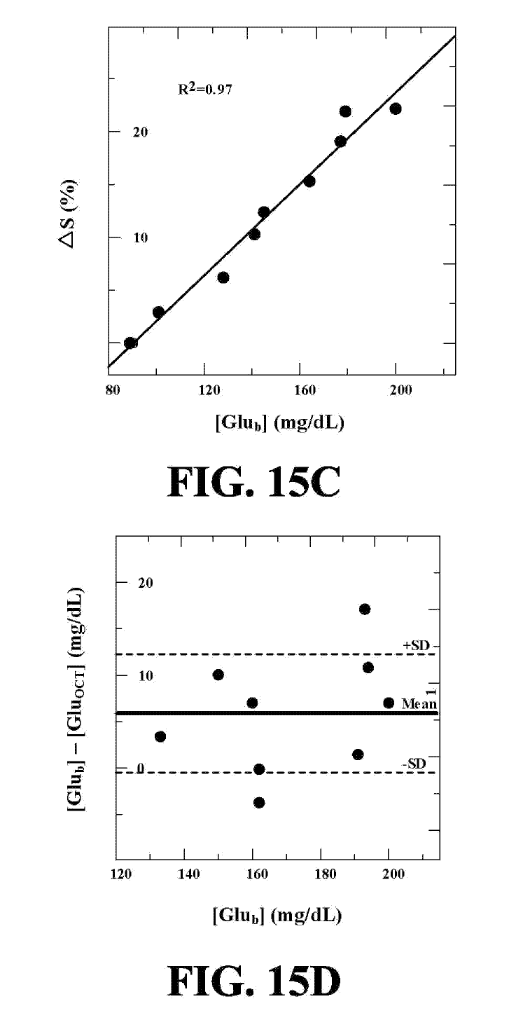

The inventors also evaluated the temperature and pressure control systems in clinical tests in healthy, non-diabetic volunteers. FIG. 15A shows the OCT signal slope measured from a forearm of a volunteer during an oral glucose tolerance test with minimal skin pressure and with and without temperature control. It is evident that, when temperature is stable, the OCT signal slope obtained from human skin in vivo closely follows [Glu_{b}].

Fourier filtering of the OCT signal slope yielded a high correlation of the OCT signal slope with [Glu_{b}] (see FIG. 15B and FIG. 15C): R²=0.97. The inventors also used the data at baseline and during increasing [Glu_{b}] (see FIG. 15C) to calibrate the OCT system and the data obtained during decreasing [Glu_{b}] to validate the accuracy of [Glu_{OCT}] calculated by using Bland-Altman analysis. FIG. 15D shows the difference between [Glu_{b}] and [Glu_{OCT}] including the standard deviation. The bias and standard deviation (±2SD) are 5.9 mg/dL (0.33 mM) and 12.8 mg/dL (0.71 mM), respectively, that closely approach clinically acceptable accuracy of 1.0 mM.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

We claim:

1. A method comprising the steps of:
generating radiation;
directing a first portion of radiation onto a location of a tissue site or a plurality of locations of a tissue site to generate backscattered radiation corresponding to a plurality of 1-D OCT signals on an intermittent, a continuous or a periodic basis under conditions of temperature and/or pressure sufficient to increase an accuracy of a calculated glucose concentration,
directing a second portion of the radiation to a reflector to generate reference radiation on a continuous or periodic basis,
combining a portion of the backscattered radiation and the reference radiation to form a combined radiation on a continuous or periodic basis,
forwarding the combined radiation to a detector to produce a plurality of optical coherence tomography signals on a continuous or periodic basis, and
calculating the glucose concentration using a single slope or a composite slope of the optical coherence tomography signals on a continuous or periodic basis,
where the number of the plurality of signals is sufficient to improve the signal-to-noise ratio of a composite OCT signal improving the OCT derived glucose concentration.

2. The method of claim 1, wherein the conditions of temperature and/or pressure comprise the step of:
maintaining a temperature of the tissue site to a temperature variation sufficient to increase an accuracy of a calculated glucose concentration.

3. The method of claim 2, wherein the temperature variation is less than about 1° C.

4. The method of claim 2, further comprising the step of:
heating the tissue site to an elevated temperature, while maintaining the temperature at the elevated temperature so that the temperature variation is less than or equal 1° C.

5. The method of claim 4, wherein the elevated temperature is between about 33° C. and 45° C.

6. The method of claim 1, wherein the conditions of temperature and/or pressure comprise the step of:
maintaining a pressure exerted on the tissue site to a minimal constant pressure sufficient to increase an accuracy of a calculated glucose concentration.

7. The method of claim 6, wherein the minimal constant pressure is less than about 0.1 kPa.

8. The method of claim 6, wherein the minimal constant pressure is less than about 0.01 kPa.

9. The method of claim 1, wherein the conditions of temperature and/or pressure comprise the step of:
maintaining a temperature of the tissue site to a temperature variation sufficient to increase an accuracy of a calculated glucose concentration, and
maintaining a pressure exerted on the tissue site to a minimal constant pressure sufficient to increase an accuracy of a calculated glucose concentration.

10. The method of claim 9, wherein the temperature variation is less than about 1° C. and the minimal constant pressure is less than about 0.1 kPa.

11. The method of claim 1, wherein the conditions of temperature and/or pressure comprise the step of:
heating the tissue site to an elevated temperature, while maintaining the tissue temperature at the elevated temperature so that a temperature variation is sufficient to increase an accuracy of a calculated glucose concentration, and
maintaining a pressure exerted on the tissue site to a minimal constant pressure is sufficient to increase an accuracy of a calculated glucose concentration.

12. The method of claim 11, wherein the elevated temperature is between about 33° C. and 45° C., the temperature variation is less than about 1° C. and the minimal constant pressure is less than about 0.1 kPa.

13. The method of claim 1, wherein the locations are within an area, where the area is regular or irregular and is between about 200 μm×200 μm and about 2000 μm×2000 μm.

14. The method of claim 13, wherein the plurality of locations comprise the entire area, a random selection of locations within the area, a patterned selection of locations within the area, a random selection of contiguous sub-areas within the area, or a patterned selection of contiguous sub-areas within the area.

15. The method of claim 1, wherein a distance between pairs of locations is between about 500 nm and 20 mm.

16. The method of claim 1, wherein each scan is an in-depth scan.

17. The method of claim 16, further comprising the step of:
constructing 2-D images of each location.

18. The method of claim 17, further comprising the step of:
constructing a 3-D image of the area from the 2-D images at each location.

19. The method of claim 1, wherein each scan is at a set tissue depth or the scans have variable tissue depths.

20. The method of claim 1, further comprising the step of:
maintaining a pressure exerted on the tissue site by an OCT probe to a minimal constant pressure sufficient to improve the accuracy of the calculated glucose concentration.

21. A method comprising the steps of:
generating first radiation having a first wavelength;
directing a first portion of first radiation onto a plurality of locations of an area of a tissue site to generate first backscattered radiation corresponding to a plurality of 1-D OCT signals on a continuous or periodic basis under conditions of temperature and/or pressure sufficient to increase an accuracy of a calculated glucose concentration,
directing a second portion of the first radiation to a reflector to generate first reference radiation on a continuous or periodic basis,
combining a portion of the first backscattered radiation and the first reference radiation to form a first combined radiation on a continuous or periodic basis,
forwarding the first combined radiation to a detector to produce a plurality of first optical coherence tomography signals on a continuous or periodic basis,
generating second radiation having a second wavelength;
directing a second portion of second radiation onto a plurality of locations of an area of a tissue site to generate second back scattered radiation corresponding to a plurality of 1-D OCT signals on a continuous or periodic basis under conditions of temperature and/or pressure sufficient to increase an accuracy of a calculated glucose concentration,
directing a second portion of the second radiation to a reflector to generate second reference radiation on a continuous or periodic basis,
combining a portion of the second backscattered radiation and the second reference radiation to form a second combined radiation on a continuous or periodic basis,
forwarding the second combined radiation to a detector to produce a plurality of second optical coherence tomography signals on a continuous or periodic basis, and
calculating a glucose concentration using data from a first composite OCT signal and a second OCT signal on a continuous or periodic basis,
where the number of the plurality of signals is sufficient to improve the signal-to-noise ratio of a composite OCT signal improving the OCT derived glucose concentration, where the first radiation is adapted to produce a high contrast OCT signal, where the second radiation is adapted to produce a water signal, and where data from the second radiation is used to reduce water artifacts during the calculating glucose concentration step.

22. The method of claim 21, wherein the first wavelength is between about 700 nm and about 1300 nm and the second wavelength is between about 1300 nm and about 2000 nm.

23. The method of claim 21, wherein the conditions of temperature and/or pressure comprise the step of:
maintaining a temperature of the tissue site to a temperature variation sufficient to increase an accuracy of a calculated glucose concentration.

24. The method of claim 23, wherein the temperature variation is less than about 1° C.

25. The method of claim 23, further comprising the step of:
heating the tissue site to an elevated temperature, while maintaining the temperature at the elevated temperature so that the temperature variation is less than or equal 1° C.

26. The method of claim 25, wherein the elevated temperature is between about 33° C. and 45° C. and the temperature variation is less than about 1° C.

27. The method of claim 21, wherein the conditions of temperature and/or pressure comprise the step of:
maintaining a pressure exerted on the tissue site to a minimal constant pressure sufficient to increase an accuracy of a calculated glucose concentration.

28. The method of claim 27, wherein the minimal constant pressure is less than 0.1 kPa.

29. The method of claim 27, wherein the minimal constant pressure is less than 0.01 kPa.

30. The method of claim 21, wherein the conditions of temperature and/or pressure comprise the step of:
maintaining a temperature of the tissue site to a temperature variation sufficient to increase an accuracy of a calculated glucose concentration, and
maintaining a pressure exerted on the tissue site to a minimal constant pressure sufficient to increase an accuracy of a calculated glucose concentration.

31. The method of claim 30, wherein the temperature variation is less than 1° C. and the minimal constant pressure is less than 0.1 kPa.

32. The method of claim 21, wherein the conditions of temperature and/or pressure comprise the step of:
heating the tissue site to an elevated temperature, while maintaining the tissue temperature at the elevated temperature so that a temperature variation is sufficient to increase an accuracy of a calculated glucose concentration, and
maintaining a pressure exerted on the tissue site to a minimal constant pressure is sufficient to increase an accuracy of a calculated glucose concentration.

33. The method of claim 32, wherein the elevated temperature is between about 33° C. and 45° C., the temperature variation is less than about 1° C. and the minimal constant pressure is less than about 0.1 kPa.

34. A method comprising the steps of:
generating radiation having a first wavelength and a second wavelength;
directing a first portion of radiation onto a plurality of locations of an area of a tissue site to generate backscattered radiation corresponding to a plurality of 1-D OCT signals on a continuous or periodic basis, while maintaining a temperature of the tissue site to a temperature variation sufficient to increase an accuracy of a calculated glucose concentration,
directing a second portion of the radiation to a reflector to generate first reference radiation on a continuous or periodic basis,
combining a portion of the backscattered radiation and the reference radiation to form a first combined radiation on a continuous or periodic basis,
forwarding the combined radiation to a detector to produce a plurality of optical coherence tomography signals on a continuous or periodic basis,
calculating a glucose concentration using data from a first composite OCT signal and a second OCT signal on a continuous or periodic basis,
where the number of the plurality of signals is sufficient to improve the signal-to-noise ratio of a composite OCT signal improving the OCT derived glucose concentration, where the first radiation is adapted to produce a high contrast OCT signal, where the second radiation is adapted to produce a water signal, and where data from the second radiation is used to reduce water artifacts during the calculating glucose concentration step.

35. The method of claim 34, where in the first wavelength is between about 700 nm and about 1300 nm and the second wavelength is between about 1300 nm and about 2000 nm.

36. The method of claim 34, wherein the conditions of temperature and/or pressure comprise the step of:
maintaining a temperature of the tissue site to a temperature variation sufficient to increase an accuracy of a calculated glucose concentration.

37. The method of claim 36, wherein the temperature variation is less than about 1° C.

38. The method of claim 36, further comprising the step of:
heating the tissue site to an elevated temperature, while maintaining the temperature at the elevated temperature so that the temperature variation is less than or equal 1° C.

39. The method of claim 38, wherein the elevated temperature is between about 33° C. and 45° C. and the temperature variation is less than about 1° C.

40. The method of claim 34, wherein the conditions of temperature and/or pressure comprise the step of:
maintaining a pressure exerted on the tissue site to a minimal constant pressure sufficient to increase an accuracy of a calculated glucose concentration.

41. The method of claim 40, wherein the minimal constant pressure is less than about 0.1 kPa.

42. The method of claim 49, wherein the minimal constant pressure is less than about 0.01 kPa.

43. The method of claim 35, wherein the conditions of temperature and/or pressure comprise the step of:
maintaining a temperature of the tissue site to a temperature variation sufficient to increase an accuracy of a calculated glucose concentration, and maintaining a pressure exerted on the tissue site to a minimal constant pressure sufficient to increase an accuracy of a calculated glucose concentration.

44. The method of claim 43, wherein the temperature variation is less than about 1° C. and the minimal constant pressure is less than about 0.1 kPa.

45. The method of claim 35, wherein the conditions of temperature and/or pressure comprise the step of:
heating the tissue site to an elevated temperature, while maintaining the tissue temperature at the elevated temperature so that a temperature variation is sufficient to increase an accuracy of a calculated glucose concentration, and
maintaining a pressure exerted on the tissue site to a minimal constant pressure is sufficient to increase an accuracy of a calculated glucose concentration.

46. The method of claim 44, wherein the elevated temperature is between about 33° C. and 45° C., the temperature variation is less than about 1° C. and the minimal constant pressure is less than about 0.1 kPa.

47. The method of claim 1, further comprising the step of:
prior to the calculating step, filtering the OCT data with a filtering routine to produce filtered OCT data.

48. The method of claim 21, further comprising the step of: prior to the calculating step, filtering the OCT data with a filtering routine to produce filtered OCT data.

49. The method of claim 34, further comprising the step of:
prior to the calculating step, filtering the OCT data with a filtering routine to produce filtered OCT data.
